(19) United States
(12) Patent Application Publication
(10) Pub. No.: US 2025/0077722 A1
(43) Pub. Date: Mar. 6, 2025

(54) CALCULATION APPARATUS, METHOD AND PROGRAM

(71) Applicant: Rigaku Corporation, Tokyo (JP)

(72) Inventors: Masatsugu YOSHIMOTO, Tokyo (JP); Kazuhiko OMOTE, Tokyo (JP)

(73) Assignee: Rigaku Corporation, Tokyo (JP)

(21) Appl. No.: 18/817,282

(22) Filed: Aug. 28, 2024

(30) Foreign Application Priority Data
Sep. 4, 2023 (JP) 2023-143063

(51) Int. Cl. G06F 30/13

(52) U.S. Cl. CPC G06F 30/13

(57) ABSTRACT

A calculation apparatus for calculating a statistics property of a structural model comprises a pre-calculation structural model acquiring section for acquiring a pre-calculation structural model in which atoms are periodically arranged, a pre-calculation information acquiring section for acquiring pre-calculation information that specifies atoms periodically arranged in the pre-calculation structural model, a post-calculation structural model acquiring section for acquiring a post-calculation structural model calculated by using the pre-calculation structural model as source data, a post-calculation information acquiring section for acquiring cluster information obtained by clustering atoms in the post-calculation structural model based on the pre-calculation information, and atom position information that is position information of atoms in the post-calculation structural model, and a statistics property calculating section for calculating centroid positions of atoms having the same cluster information based on the cluster information and the atom position information.

### CROSS-REFERENCE TO RELATED APPLICATION

This application claims priority from Japanese Patent Application No. 2023-143063 filed on Sep. 4, 2023, the entire contents of Japanese Patent Application No. 2023-143063 are incorporated herein by reference.

### BACKGROUND

### Field

The present disclosure relates to a calculation apparatus, method, and program for calculating a statistics property of a structural model.

### Description of the Related Art

In recent years, the properties of materials such as batteries and electronics are closely related to the local structure of the material. Therefore, the estimation of the local structure model (structural model) which reproduces the measured values is crucial, and this problem has been solved using, for example, Reverse Monte Carlo (RMC).

For a deep understanding of the function of materials, it is essential to extract statistics properties from the estimated structural model. However, extracting the statistics property from the estimated structural model depends on the proficiency of the analysis, and it is difficult to provide useful information to all analysts.

Non-Patent Document 1 discloses analyzing the local structure of Ga atoms in a γ-Ga₂O₃ by modeling the neutron total scattering data by a Reverse Monte Carlo. As a method of displaying the modeling result by the Reverse Monte Carlo, a degenerate structural modeling which is a method of directly displaying the distribution of atoms by folding the calculation cell back to the original unit cell has been reported.

Non-Patent Document 2 discloses modeling SrTiO₃ neutron total scattering data by the Reverse Monte Carlo in order to examine a device suitable for the Reverse Monte Carlo. As a method of displaying the probability density distribution of O atoms in a structural model after modeling, a method of displaying a projection view from a specific lattice plane is reported.

Non-Patent Document 3 discloses analyzing the distribution of cations by modeling by the Reverse Monte Carlo on the neutron and X-ray total scattering data of the ceria-zirconia-based composite oxide. As a method of displaying a structural model after modeling, a method similar to that of Non-Patent Document 1 is used. Further, as a method of representing the displacement amounts of atoms, a method of displaying the displacement amount from the initial structure in a histogram has been reported.

### Non-Patent Document

Non-patent Document 1: Helen. Y. Playford et al., J. Phys. Chem. C. 118(2014) 16188-16198.

Non-patent Document 2: Y. Zhang, M. Eremenko, V. Krayzman, M. G. Tucker, I. Levin, J. Appl. Crystallography 53(2020) 1509-1518.

Non-patent Document 3: A. Summer et al., APL Materials. 11(2023) 031113.

### SUMMARY

FIGS. 1A and 1B are examples of a structural model and a degenerate structural model thereof, respectively. The degenerate structural model in FIG. 1B is magnified. Thus, the qualitative features of the structural model can be visually confirmed by converting the structural model into a degenerate structural model. On the other hand, as a quantitative statistics property of the structural model, the centroid position of each atom in the degenerate structural model is an important value. However, none of Non-Patent Document 1 to Non-Patent Document 3 takes into account of calculating the centroid position, and such a value cannot be calculated heretofore.

As a result of intensive studies, the present inventors have found that the centroid position of each atom in the degenerate structural model can be calculated by enabling information on the position of each atom before and after simulation to be acquired.

The present disclosure has been made in view of such circumstances, and an object of the present disclosure is to provide a calculation apparatus, method and program for calculating a statistics property of a structural model.

(1) Accordingly, the calculation apparatus of the present disclosure is a calculation apparatus for calculating a statistics property of a structural model, a pre-calculation structural model acquiring section for acquiring a pre-calculation structural model in which atoms are periodically arranged, a pre-calculation information acquiring section for acquiring pre-calculation information that specifies atoms periodically arranged in the pre-calculation structural model, a post-calculation structural model acquiring section for acquiring a post-calculation structural model calculated by using the pre-calculation structural model as source data, a post-calculation information acquiring section for acquiring cluster information obtained by clustering atoms in the post-calculation structural model based on the pre-calculation information, and atom position information that is position information of atoms in the post-calculation structural model, and a statistics property calculating section for calculating centroid positions of atoms having the same cluster information based on the cluster information and the atom position information.

(2) Further, the calculation apparatus of the present disclosure further comprises a simulation executing section for calculating the post-calculation structural model using the pre-calculation structural model as source data, wherein the post-calculation structural model acquiring section acquires the post-calculation structural model calculated by the simulation executing section.

(3) Further, in the calculation apparatus of the present disclosure, the simulation executing section calculates the post-calculation structural model by using RMC or MD.

(4) Further, the calculation apparatus further comprises a simulation condition setting section for setting a size of a calculation cell in RMC or MD.

(5) Further, in the calculation apparatus of the present disclosure, the pre-calculation information is information of atomic coordinates in the pre-calculation structural model.

(6) Further, in the calculation apparatus of the present disclosure, the statistics property calculating section calculates, in addition to the centroid position, displacement information regarding displacements of atoms having the same cluster information from the centroid positions based on the cluster information and the atom position information.

(7) Further, the calculation apparatus of the present disclosure further comprises a degenerate structural model displaying section for displaying a degenerate structural model in which a unit structural model calculated by dividing the post-calculation structural model based on periodic information of the pre-calculation structural model is superimposed, and a statistics property displaying section for displaying a statistics property on the degenerate structural model, wherein the statistics property displayed on the degenerate structural model by the statistics property displaying section is the centroid position.

(8) Further, the calculation apparatus of the present disclosure further comprises a degenerate structural model displaying section for displaying a degenerate structural model in which a unit structural model calculated by dividing the post-calculation structural model based on periodic information of the pre-calculation structural model is superimposed, and a statistics property displaying section for displaying a statistics property on the degenerate structural model, wherein the statistics property displayed on the degenerate structural model by the statistics property displaying section is the displacement information.

(9) Further, in the calculation apparatus of the present disclosure, the degenerate structural model displaying section displays a plurality of the degenerate structural models in comparison, and the statistics property displaying section displays the statistics properties corresponding to the respective degenerate structural models on them.

(10) Further, in the calculation apparatus of the present disclosure, the degenerate structural model displaying section displays a plurality of the degenerate structural models in comparison, the statistics property displaying section displays the statistics properties corresponding to the respective degenerate structural models, and the statistics properties displayed on the respective degenerate structural models by the statistics property displaying section is the centroid position or the displacement information.

(11) Further, the method of the present disclosure is a method for calculating a statistics property of a structural model, and comprises the steps of acquiring pre-calculation information that specifies atoms periodically arranged, acquiring pre-calculation information for specifying atoms arranged periodically in the pre-calculation structural model, acquiring a post-calculation structural model calculated by using the pre-calculation structural model as source data, acquiring cluster information obtained by clustering atoms in the post-calculation structural model based on the pre-calculation information, and atom position information that is position information of atoms in the post-calculation structural model, and calculating centroid positions of atoms having the same cluster information based on the cluster information and the atom position information.

(12) Further, the program of the present disclosure causes a computer to execute the process of acquiring a pre-calculation structural model in which atoms are periodically arranged, acquiring pre-calculation information that specifies atoms periodically arranged in the pre-calculation structural model, acquiring a post-calculation structural model calculated by using the pre-calculation structural model as source data, acquire cluster information obtained by clustering atoms in the post-calculation structural model based on the pre-calculation information, and acquire atom position information that is position information of atoms in the post-calculation structural model, and acquiring cluster information obtained by clustering atoms in the post-calculation structural model based on the pre-calculation information, and atom position information that is position information of atoms in the post-calculation structural model, and calculating centroid positions of atoms having the same cluster information based on the cluster information and the atom position information.

### BRIEF DESCRIPTION OF THE DRAWINGS

FIG. 1A and FIG. 1B are examples of a structural model and a degenerate structural model thereof, respectively.

FIG. 2 is a block diagram showing an example of the configuration of the calculation apparatus.

FIG. 3 is a block diagram showing a modified example of the configuration of the calculation apparatus.

FIG. 4 is a block diagram showing a modified example of the configuration of the calculation apparatus.

FIG. 5 is a block diagram showing a modified example of the configuration of the calculation apparatus.

FIG. 6 is a block diagram showing a modified example of the configuration of the calculation apparatus.

FIG. 7 is a flowchart showing an example of the operation of the calculation apparatus.

FIG. 8 is a flowchart showing a modified example of the operation of the calculation apparatus.

FIG. 9 is a flowchart showing a modified example of the operation of the calculation apparatus.

FIG. 10 is a schematic diagram showing an example of the configuration of the calculation system for X-ray diffraction measurement.

FIG. 11 is a block diagram showing an example of the configuration of the control apparatus and the calculation apparatus.

FIG. 12 is a block diagram showing a modified example of the configurations of the control apparatus and the calculation apparatus.

FIG. 13A and FIG. 13B are diagrams showing statistics properties superimposed on the degenerate structural model of example 1, respectively.

FIG. 14 is a histogram showing the distribution of the average displacement amounts from the centroid positions of all atoms in example 1.

FIG. 15 is a diagram showing statistics properties superimposed on the degenerate structural model of example 2.

FIG. 16 is a table showing the average displacement amounts from the centroid positions of the atoms at the respective sites of example 2 and the standard deviations of the average displacement amounts.

FIG. 17A and FIG. 17B are diagrams showing statistics properties superimposed on the degenerate structural model of example 3, respectively.

FIG. 18 is a table showing the average displacement amounts from the centroid positions of the atoms at respective sites of example 3 and the standard deviation of the average displacement amounts.

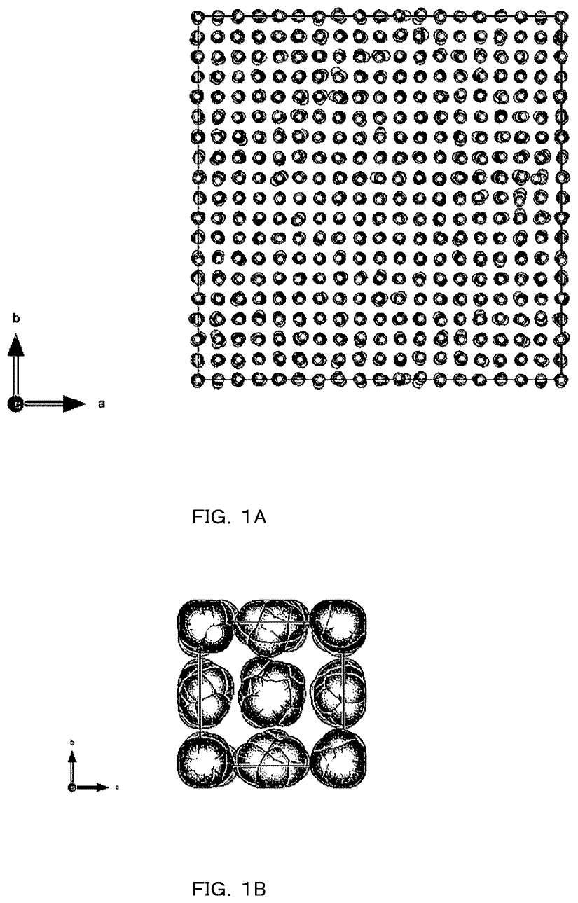

### DETAILED DESCRIPTION

Next, embodiments of the present disclosure are described with reference to the drawings. To facilitate understanding of the description, the same reference numerals are assigned to the same components in the respective drawings, and duplicate descriptions are omitted.

### Embodiment

### [Calculation Apparatus]

The calculation apparatus 100 is configured by a computer formed by connecting a CPU (Central Processing Unit), ROM (Read Only Memory), RAM (Random Access Memory) and a memory to a bus. The calculation apparatus 100 may be a PC terminal or a server on a cloud. Not only the whole apparatus but also part of the apparatus or some functions of the apparatus may be provided on the cloud. For example, the calculation apparatus 100 may be connected to a measurement apparatus such as the X-ray diffraction apparatus 200 via a control apparatus 300, which is described later.

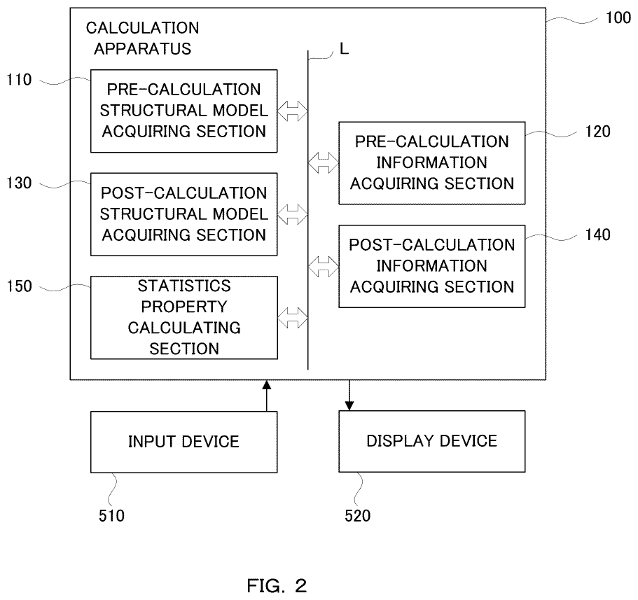

The calculation apparatus 100 calculates statistics properties of a structural model. The statistics properties of the structural model and the statistics properties of the degenerate structural model obtained by converting the structural model may be regarded as the same. FIG. 2 is a block diagram showing an example of the configuration of the calculation apparatus 100. The calculation apparatus 100 comprises a pre-calculation structural model acquiring section 110, a pre-calculation information acquiring section 120, a post-calculation structural model acquiring section 130, a post-calculation information acquiring section 140, and a statistics property calculating section 150. Each section can transmit and receive information via the control bus L. The input device 510 and the display device 520 are connected to CPU of the calculation apparatus 100 via an appropriate interface. The input device 510 is, for example, a keyboard or a mouse and performs input to the calculation apparatus 100. The display device 520 is, for example, a display and displays a pre-calculation structural model, pre-calculation information, a post-calculation structural model, cluster information, atom position information, a statistics property or the like.

The pre-calculation structural model acquiring section 110 acquires a pre-calculation structural model in which atoms are periodically arranged. The structural model is data indicating an atomic arrangement in a finite region and indicates, for example, an arrangement of finite number of atoms in a cube, cuboid, or parallel hexahedron. The pre-calculation structural model is data of an atomic arrangement based on a crystal structure or the like of a sample for measuring measurement data.

The structural model to which the present disclosure can be applied may be generated based on measurement data of any apparatus as long as it is a structural model generated by structural modeling in which a crystal structure is an initial structure. For example, the present disclosure is not limited to a structural model generated based on total scattering data measured by an X-ray diffraction apparatus and can be applied to a structural model generated based on measurement data measured by a probe similar thereto. Specifically, the present disclosure can be applied to a structural model generated on the basis of, for example, measurement data by synchrotron radiation and measurement data by particle beams such as neutron beams and electron beams.

The pre-calculation structural model acquiring section 110 may acquire the pre-calculation structural model from an external apparatus that performs the simulation when the calculation apparatus 100 does not perform the simulation for calculating a post-calculation structural model to be described later using the pre-calculation structural model as the source data. In addition, when the simulation executing section 160, which is described later, of the calculation apparatus 100 performs a simulation for calculating the post-calculation structural model using the pre-calculation structural model as the source data, the pre-calculation structural model acquiring section 110 may acquire the pre-calculation structural model from the simulation executing section 160. The acquisition of the pre-calculation structural model may be the designation or input of data from the user to the calculation apparatus 100. The same applies to the subsequent acquisition of the information of the calculation apparatus 100.

The pre-calculation information acquiring section 120 acquires the pre-calculation information specifying periodically arranged atoms in the pre-calculation structural model.

The pre-calculation information may be any information that specifies individual atoms. For example, the pre-calculation information may be information of atomic coordinates in the pre-calculation structural model, or may be numbers assigned to atoms.

The information of the atomic coordinates in the pre-calculation structural model may be atomic coordinates (fractional coordinates, actual coordinates or the like) indicating positions in the entire pre-calculation structural model. Further, the information of the atomic coordinates in the pre-calculation structural model may be a set of values indicating the position of an atom in the pre-calculation unit structural model (cell, unit cell) calculated by dividing the pre-calculation structural model based on the periodic information of the pre-calculation structural model and a cell number indicating the position in the pre-calculation structural model of the pre-calculation unit structural model. The values indicating the positions of the atoms in the pre-calculation unit structural model may be atomic coordinates (fractional coordinates, actual coordinates or the like) in the pre-calculation unit structural model or may be numbers assigned to sites indicating the positions of the atoms in the pre-calculation unit structural model.

The numbers assigned to the atoms may be separately stored as a table for specifying the positions of the atoms of the numbers in the entire pre-calculation structural model.

The post-calculation structural model acquiring section 130 acquires the post-calculation structural model calculated using the pre-calculation structural model as the source data. The post-calculation structural model acquiring section 130 may acquire the post-calculation structural model from an external apparatus that performs the simulation when the calculation apparatus 100 does not perform the simulation of calculating the post-calculation structural model using the pre-calculation structural model as the source data. Further, when the simulation executing section 160 of the calculation apparatus 100 performs a simulation for calculating the post-calculation structural model using the pre-calculation structural model as the source data, the post-calculation structural model acquiring section 130 may acquire the post-calculation structural model from the simulation executing section 160.

The post-calculation information acquiring section 140 acquires cluster information obtained by clustering atoms in the post-calculation structural model on the basis of pre-calculation information and atom position information which is atom position information in the post-calculation structural model. The information acquired by the post-calculation information acquiring section 140 may be cluster information or atom position information itself or may be information necessary for calculating cluster information or atom position information. When the cluster information is not directly obtained from the pre-calculation information or the atom position information is not directly obtained from the post-calculation structural model, the post-calculation information acquiring section 140 may acquire the cluster information or the atom position information calculated by an external apparatus. Further, the post-calculation information acquiring section 140 may calculate the cluster information or the atom position information. In the case where the post-calculation information acquiring section 140 acquires the cluster information and the atom position information calculated by the external apparatus and does not comprise the degenerate structural model displaying section 170, which is described later, the post-calculation structural model acquiring section 130 may not acquire the post-calculation structural model.

The cluster information is information obtained by clustering atoms that are recognized as being at the same site in the pre-calculation unit structural model by the pre-calculation information as belonging to the same cluster. Thus, the cluster information may be values indicating the positions of atoms in the pre-calculation unit structural model. The cluster information may be atomic coordinates (fractional coordinates, actual coordinates or the like) in the pre-calculation unit structural model or may be numbers assigned to sites indicating the positions of the atoms in the pre-calculation unit structural model.

The atom position information in the post-calculation structural model may be atomic coordinates (fractional coordinates, actual coordinates or the like) indicating positions in the entire post-calculation structural model. The atom position information in the post-calculation structural model may be a set of values indicating the former positions of the atoms and coordinates (vectors) indicating the movement amounts and directions from the former positions in the post-calculation structural model. The values indicating the former positions of the atoms in the post-calculation structural model may be information of atomic coordinates in the pre-calculation information or may be numbers assigned to the atoms. These details are as described above.

The statistics property calculating section 150 calculates the centroid position of an atom having the same cluster information based on the cluster information and the atom position information. The centroid position can be calculated by averaging the atomic coordinates of the calculated unit structural model of atoms having the same cluster information.

The statistics property calculating section 150 may calculate displacement information regarding displacement from the centroid position of an atom having the same cluster information based on the cluster information and the atom position information in addition to the centroid position. The displacement information regarding the displacement from the centroid position is, for example, the Volume data of the density distribution from the centroid position, the average displacement from the centroid positions by sites, the standard deviation thereof or the like. It is also possible to calculate the average displacement from the centroid position of each atom or the standard deviation thereof from the average displacement from the centroid position at each site or the standard deviation thereof. Therefore, the average displacement from the centroid position of each atom and the standard deviation thereof are also included in the displacement information.

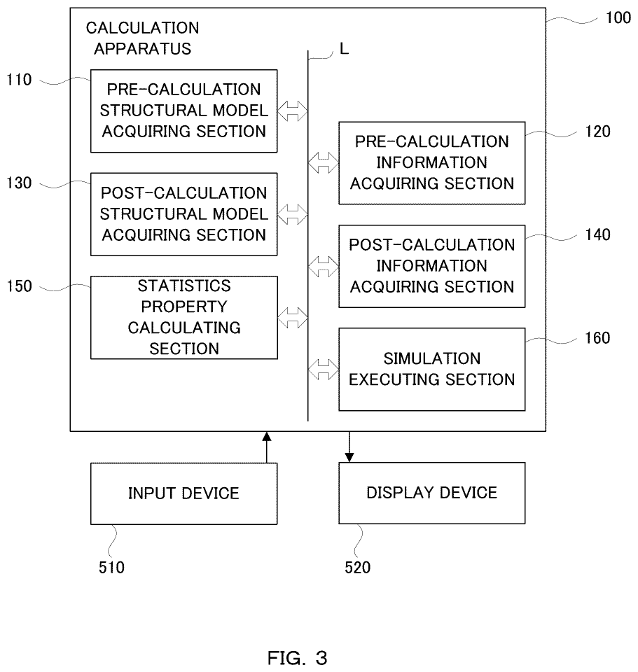

FIG. 3 is a block diagram showing a modified example of the configuration of the calculation apparatus 100. As shown in FIG. 3, the calculation apparatus 100 may comprise a simulation executing section 160. The simulation executing section 160 calculates the post-calculation structural model using the pre-calculation structural model as the source data. The post-calculation structural model is a structural model simulated to account for measured data measured with a probe such as an X-ray, synchrotron radiation, neutron, or electron beam. The simulation executing section 160 may secure a calculation region based on the size, shape, atomic arrangement, and the like of the pre-calculation structural model and calculate the post-calculation structural model. The size, shape, initial atomic arrangement and the like of the structural model may be designated by the user. The simulation executing section 160 executes the simulation so that the pre-calculation information is held. For example, the simulation is performed while distinguishing each atom so that the number assigned to each atom before the simulation can be confirmed even after the simulation. The same applies to a case where an external apparatus performs a simulation.

When the calculation apparatus 100 comprises the simulation executing section 160, the post-calculation structural model acquiring section acquires the post-calculation structural model calculated by the simulation executing section 160. When the calculation apparatus 100 comprises the simulation executing section 160, the pre-calculation information acquiring section 120 may acquire the measurement data as pre-calculation information.

The simulation executing section may calculate the post-calculation structural model by RMC (Reverse Monte Carlo) or MD (Molecular Dynamics). RMC is a method for estimating a structural model that reproduces actual measured values by moving the atomic arrangement of a structural model using random numbers. The RMC has a wide search space and can obtain a global minimum solution, which is useful as a solution for complicated optimization. Furthermore, Molecular Dynamics (MD) is a method for obtaining time variation in position data of each atom by obtaining the force acting on each atom due to potential energy and solving Newton's equation of motion.

When RMC or MD is applied, there is a high possibility that a structural model (post-calculation structural model) that reproduces measurement data is obtained, and there is a high possibility that a statistics property such as a centroid position calculated based on the structural model becomes a significant value for understanding the measurement data.

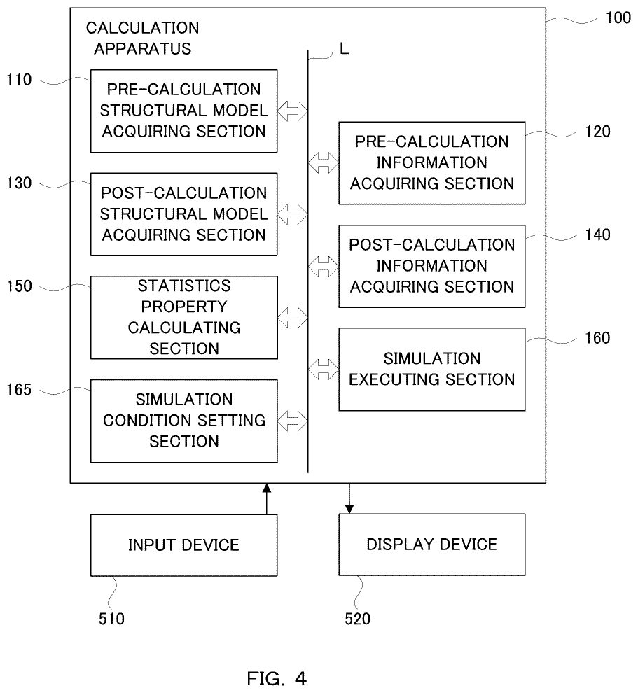

FIG. 4 is a block diagram showing a modified example of the configuration of the calculation apparatus 100. As shown in FIG. 4, when the calculation apparatus 100 comprises the simulation executing section 160, the calculation apparatus may comprise the simulation condition setting section 165. The simulation condition setting section 165 sets the size of the calculation cell in RMC or MD. Since the accuracy of the post-calculation structural model and the calculation time have a trade-off relationship with each other, the size of the calculation cell is set to an appropriate size in accordance with the targeted accuracy of the post-calculation structural model. The simulation condition setting section 165 may set the size of the calculation cell based on a value such as a calculation time specified by the user.

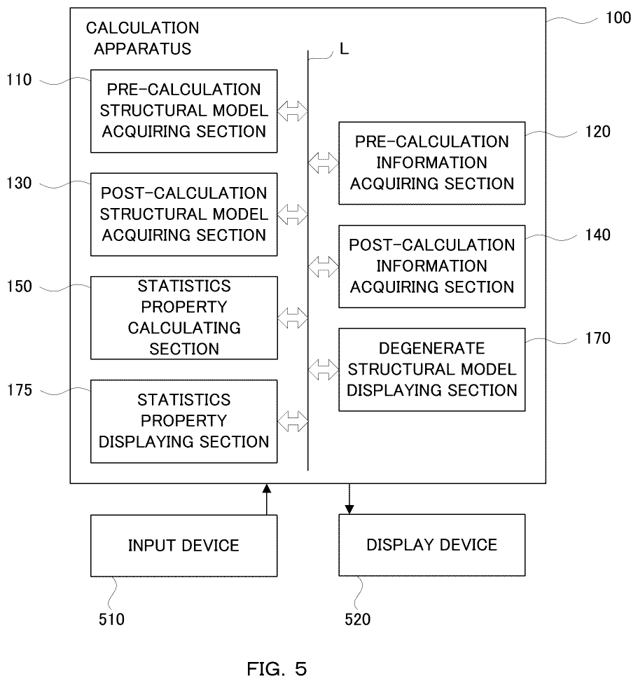

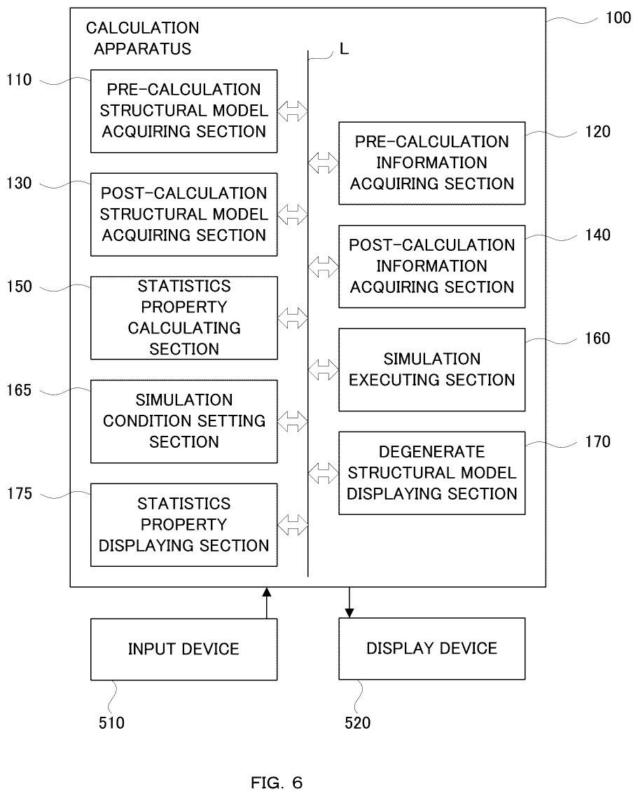

FIG. 5 or FIG. 6 is a block diagram showing a modified example of the configuration of the calculation apparatus 100. As shown in FIG. 5 or FIG. 6, the calculation apparatus 100 may comprise a degenerate structural model displaying section 170 and a statistics property displaying section 175. The degenerate structural model displaying section 170 displays a degenerate structural model obtained by superimposing the unit structural models calculated by dividing the post-calculation structural model based on the periodic information of the pre-calculation structural model. The degenerate structural model to be displayed may be a two-dimensional image or a three-dimensional image. In addition, the display may be a moving image in which the viewing direction of the degenerate structural model changes.

The statistics property displaying section 175 displays the statistics properties on the degenerate structural model. The statistics property to be displayed may be a centroid position. Further, the statistics properties to be displayed may be displacement information related to displacement from the centroid position of atom having the same cluster information. The displacement information regarding the displacement from the centroid positions includes, for example, the Volume data of the density distribution from the centroid positions, the average displacement from the centroid positions at respective sites, the standard deviation thereof, the average displacement from the centroid positions of respective atoms, the standard deviation thereof or the like.

The statistics property displaying section 175 may display a plurality of statistics properties superimposed on one degenerate structural model. The statistics property displaying section 175 may display only statistics properties for a part of atoms or sites. For example, by extracting only the density distribution of ions that are likely to migrate, the paths of ion conductors in a solid electrolyte or the like can be visualized. As a result, it is possible to contribute to understanding the properties of batteries using a solid electrolyte, materials used in fuel cells or the like. The statistics property displaying section 175 may display only the statistics properties without displaying some or all atoms of the degenerate structural model. Such a case is also included in the case that the statistics properties are displayed on the degenerate structural model. The statistics property displaying section 175 may display the statistics properties with a table or a graph. In the block diagram of FIG. 3, a degenerate structural model displaying section 170 and a statistics property displaying section 175 may be further provided.

The degenerate structural model displaying section 170 may display a plurality of degenerate structural models in comparison with each other. In this case, the statistics property displaying section 175 may display the corresponding statistics properties on each degenerate structural model. Displaying a plurality of degenerate structural models in comparison includes displaying a plurality of degenerate structural models on one screen, automatically or manually switching a screen on which the degenerate structural model of 1 is displayed or the like. The degenerate structural model to be displayed in contrast may be a degenerate structural model generated from the same data or may be a degenerate structural model generated from different data. The corresponding statistics properties displayed on the respective degenerate structural models may be the same statistics properties or different statistics properties. For example, different statistics properties can be displayed to be compared on the same degenerate structural model. In addition, for example, a plurality of degenerate structural models and statistics properties respectively generated by the data measured under the shifted measurement conditions can be serially displayed in a moving image.

In this way, the statistics properties of the structural model can be calculated. In addition, the statistics properties can be superimposed on the degenerate structural model and can be confirmed.

### Calculation Method of Centroid Position

### (Description 1 of Flow Until Centroid Position Calculation)

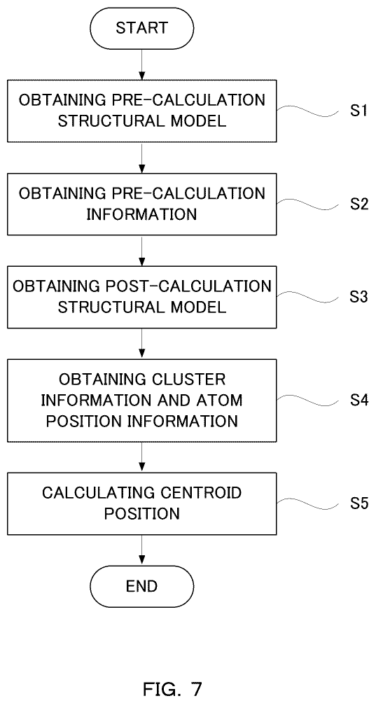

FIG. 7 is a flowchart showing an example of the operation of the calculation apparatus 100. FIG. 7 shows an example of an operation until the centroid positions are calculated based on the simulation performed by an external apparatus for a structural model. First, the calculation apparatus 100 obtains a pre-calculation structural model (step S1). Next, pre-calculation information is acquired (step S2). Next, a post-calculation structural model is acquired (step S3). Next, cluster information and atom position information are acquired (step S4). Then, the centroid positions are calculated (S5 in steps), and the process ends. After the calculation of the centroid positions, the displacement information may be further calculated. Further, the post-calculation structural model, the centroid positions, or the displacement information may be output as necessary. In this way, the centroid position can be calculated from the cluster information and the atom position information.

In this flow, since the simulation of the structural model is performed by an external apparatus, the acquisition of the pre-calculation structural model, the acquisition of the pre-calculation information, and the acquisition of the post-calculation structural model may be performed in different order or simultaneously. In addition, in a case where the calculation of the cluster information and the atom position information is performed by an external apparatus, the acquisition of the pre-calculation structural model, the acquisition of the pre-calculation information, the acquisition of the post-calculation structural model, and the acquisition of the cluster information and the atom position information may be performed in different order or simultaneously. In the following flowcharts, the order in which the information is acquired may be changed as long as the statistics properties such as the centroid positions can be calculated.

### (Description 2 of Flow Until Centroid Position Calculation)

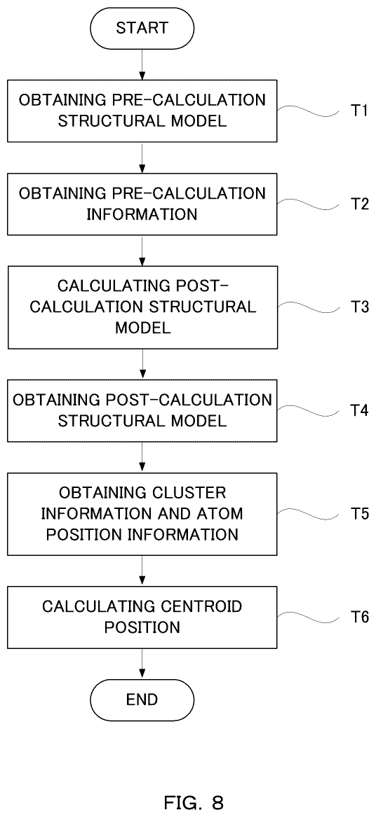

FIG. 8 is a flowchart showing a modified example of the operation of the calculation apparatus 100. FIG. 8 shows an example of the operation in the case that the calculation apparatus 100 also performs a simulation of the structural model. In the following description of the flowchart, the characteristic operation is described in detail, and the description of the operation already described may be omitted. The acquisition of the pre-calculation structural model (step T1) and the acquisition of the pre-calculation information (step T2) are similar to the above-described step S1 and step S2. Next, the calculation apparatus 100 calculates the post-calculation structural model (step T3). The post-calculation structural model is calculated as a structural model making the measurement data interpretable using the pre-calculation structural model as the source data.

Next, the processes from the acquisition of the post-calculation structural model (step T4) to the calculation of the centroid position (step T6) are the same as those from the step S3 to step S5 described above, and the operation is finished after the calculation of the centroid position. Thus, the calculation apparatus 100 can perform the calculation including the calculation of the post-calculation structural model, and the series of processes can be performed regardless of the external apparatus.

### (Description of Flow to Displaying Statistics Properties in Degenerate Structural Model)

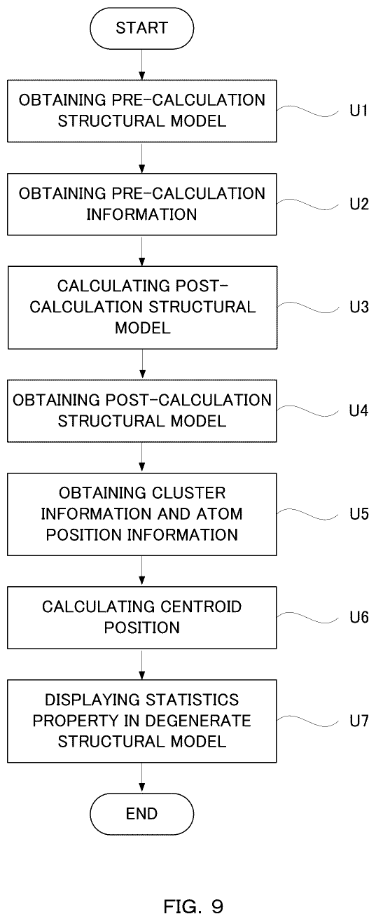

FIG. 9 is a flowchart showing a modified example of the operation of the calculation apparatus 100. FIG. 9 shows an example of the operation until the statistics properties are displayed in the degenerate structural model. The processes from the acquisition of the pre-calculation structural model (step U1) to the calculation of the centroid position (step U6) are the same as those from the above-described step T1 to step T6. Then, the statistics properties are displayed on the degenerate structural model (step U7). First, the post-calculation structural model is divided into post-calculation unit structural models based on the periodic information of the post-calculation structural model. Next, each atom of the calculated unit structural model is represented by a point, a sphere or another appropriate shape on the screen and displayed in a superimposed manner to display the degenerate structural model. Then, the statistics properties are superimposed on the degenerate structural model. Thus, it is possible to display statistics properties such as the centroid positions on the degenerate structural model in a superimposed manner, and it is possible to simultaneously confirm quantitative values of the statistics properties and a qualitative expression of the degenerate structural model.

### Whole System

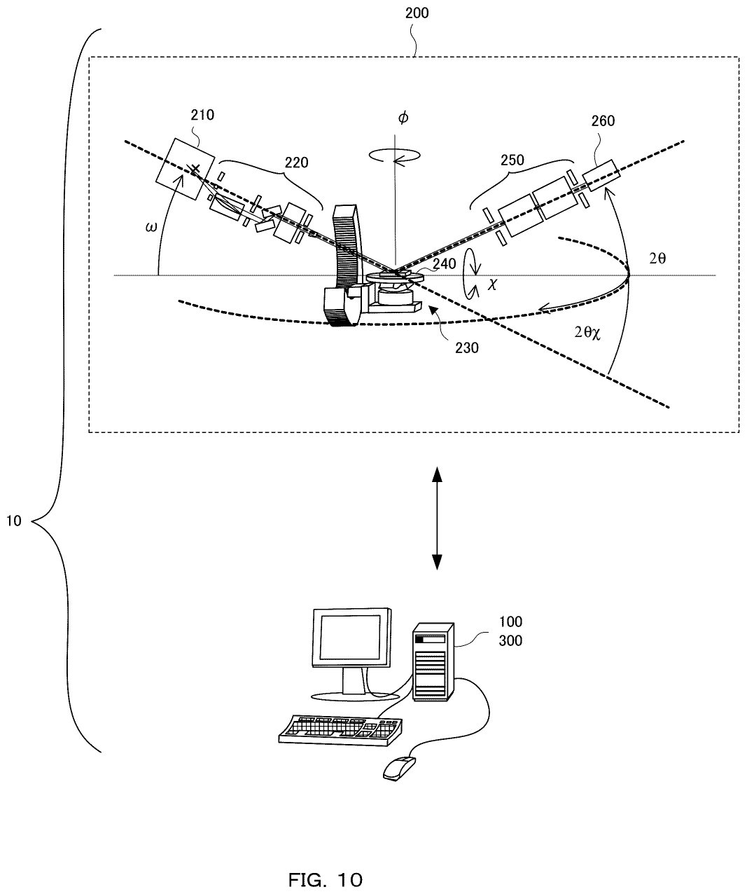

The calculation apparatus 100 of the present disclosure can be configured as a calculation system 10 comprising, for example, an X-ray diffraction apparatus. FIG. 10 is a schematic diagram showing an example of the configuration of the calculation system 10 comprising an X-ray diffraction apparatus. The calculation system 10 comprises a calculation apparatus 100, an X-ray diffraction apparatus 200 and a control apparatus 300. The X-ray diffraction apparatus 200 makes X-rays incident on a sample and constitutes an optical system for detecting diffracted X-rays generated from the sample, and the optical system comprises a goniometer. Incidentally, the configuration shown in FIGS. is one example, and thus a variety of other configurations may be adopted.

The control apparatus 300 is connected to the X-ray diffraction apparatus 200 and controls the X-ray diffraction apparatus 200 and processes and stores the acquired data. The calculation apparatus 100 is the calculation apparatus 100 described above. The control apparatus 300 and the calculation apparatus 100 are apparatuses comprising CPU and memories and may be PC terminals or servers on the cloud. Further, not only the whole apparatus but also part of the apparatus or some functions of the apparatus may be provided on the cloud. The input device 510 is, for example, a keyboard or a mouse, and performs input to the control apparatus 300. The display device 520 is, for example, a display, and displays measurement data, a pre-calculation structural model, pre-calculation information, a post-calculation structural model, cluster information, atom position information, a statistics property or the like.

By using such a calculation system 10, for example, it is possible to measure the total scattered data of a sample, estimate a structural model (post-calculation structural model) describing the total scattered data, and calculate a statistics property thereof. In addition, the statistics properties can be superimposed and displayed on the degenerate structural model. As a result, it is possible to easily calculate and confirm the statistics properties of the structural model.

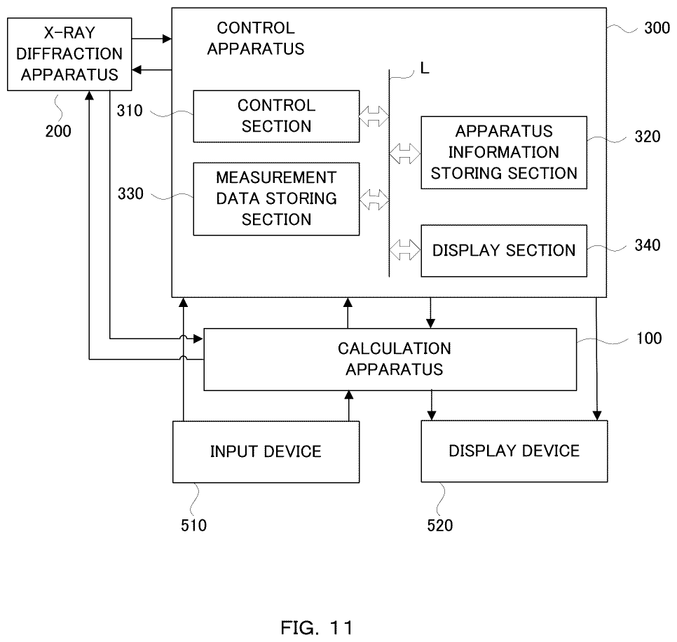

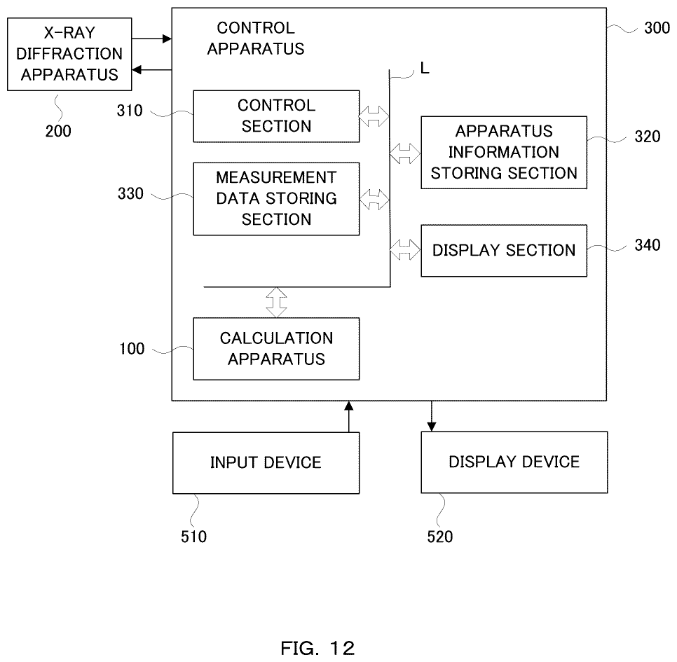

In FIG. 10, the control apparatus 300 and the calculation apparatus 100 are described as the same PC. However, as described above, the method of the present disclosure, independent of the X-ray diffractometer 200 and the control apparatus 300, the pre-calculation structural model, pre-calculation information, post-calculation structural model and atom position information can be obtained, it is possible to calculate the statistics property such as the centroid position. Therefore, as shown in FIG. 11, the calculation apparatus 100 may be configured as an apparatus different from the control apparatus 300. FIG. 11 is a block diagram showing an example of the configuration of the control apparatus 300 and the calculation apparatus 100. Further, as shown in FIG. 12, the calculation apparatus 100 may be configured for a part of functions included in the control apparatus 300. In addition, the calculation apparatus 100 and the control apparatus 300 may be configured as an integrated apparatus. FIG. 12 is a block diagram showing a modified example of the configurations of the control apparatus 300 and the calculation apparatus 100.

### X-Ray Diffraction Apparatus

The X-ray diffraction apparatus 200 comprises an X-ray generation section 210 that generates X-rays from an X-ray focus, that is, an X-ray source, an incident side optical unit 220, a goniometer 230, a sample stage 240 where a sample is set, an emitting side optical unit 250 and a detector 260 that detects X-rays. The X-ray generation section 210, the incident side optical unit 220, the goniometer 230, the sample stage 240, the emitting side optical unit 250 and the detector 260 constituting the X-ray diffraction apparatus 200 may be those generally available, and thus descriptions are omitted for them.

### Control Apparatus

The control apparatus 300 is configured from a computer formed by connecting CPU, ROM, RAM and a memory to a bus. The controller 300 is connected to the X-ray diffraction apparatus 200 and controls the operation of the X-ray diffraction apparatus 200. In addition, the control apparatus 300 receives information of the X-ray diffraction apparatus 200.

The control apparatus 300 comprises the control section 310, the apparatus information storing section 320, the measurement data storing section 330, and the display section 340. Each section can transmit and receive information via the control bus L. When the calculation apparatus 100 and the control apparatus 300 have different configurations, the input device 510 and the display device 520 are also connected to CPU of the control apparatus 300 via an appropriate interface. In this case, the input device 510 and the display device 520 may be different from those connected to the calculation apparatus 100.

The control section 310 controls the operations of the X-ray diffraction apparatus 200. The apparatus information storing section 320 stores apparatus information acquired from the X-ray diffraction apparatus 200. The apparatus information includes information about the X-ray diffraction apparatus 200 such as name of the apparatus, a kind of a radiation source, a wavelength, a background, and so forth. In addition, necessary information among information such as a shape, arrangement, type of constituent elements, composition and absorption coefficient of a sample may be included.

The measurement data storing section 330 stores the measurement data acquired from the X-ray diffraction apparatus 200. The measurement data may include, for example, total scattering data. Further, necessary information among information such as a kind of a radiation source, wavelength, background and shape, arrangement, type of constituent elements, composition, absorption coefficient of a sample and the like. The display section 340 displays the measurement data on the display device 520. Thus, the measurement data can be confirmed by the user. Further, instruction and designation to the control apparatus 300 and so forth can be made based on the measurement data by the user.

### Measurement Method

A sample S is installed in the X-ray diffraction apparatus 200, and the goniometer is driven under a predetermined condition based on the control of the control apparatus 300. Further, X-rays are incident on the sample, and diffracted X-rays generated from the sample are detected. Thus, the diffraction data is acquired. The X-ray diffraction apparatus 200 transmits the apparatus information, etc. and the acquired diffraction data as the measurement data to the control apparatus 300. With respect to such measurement data, statistics properties such as the centroid positions of respective atoms of the structural model can be calculated by the calculation apparatus 100 or the calculation method of the present disclosure.

### EXAMPLE

### Example 1

The system 10 configured as described above was used to measure the total scattering data of Ni. This was used to calculate the structure factor S(Q) of the total scattering data. Next, a pre-calculation structural model with pre-calculation information was generated, and S(Q) of the structural model was calculated. Next, the generation of the structural model is repeated by RMC until the degree of deviation between S(Q) of the total scattering data and S(Q) of the structural model becomes sufficiently small. At this time, the atomic coordinates were changed while the pre-calculation information was retained so that the pre-calculation information of each atom was recognized. The structural model when the degree of deviation satisfies a predetermined condition was used as the post-calculation structural model.

Next, using the method of the present disclosure, the cluster information was obtained by clustering atoms in the post-calculation structural model based on pre-calculation information. The atom position information is obtained from the post-calculation structural model. Then, the centroid positions of the atoms having the same cluster information were calculated based on the cluster information and the atom position information. In addition, the Volume data of the density distribution of atoms at respective sites, the average displacement from the centroid positions of all atoms and the standard deviation of the average displacement were calculated.

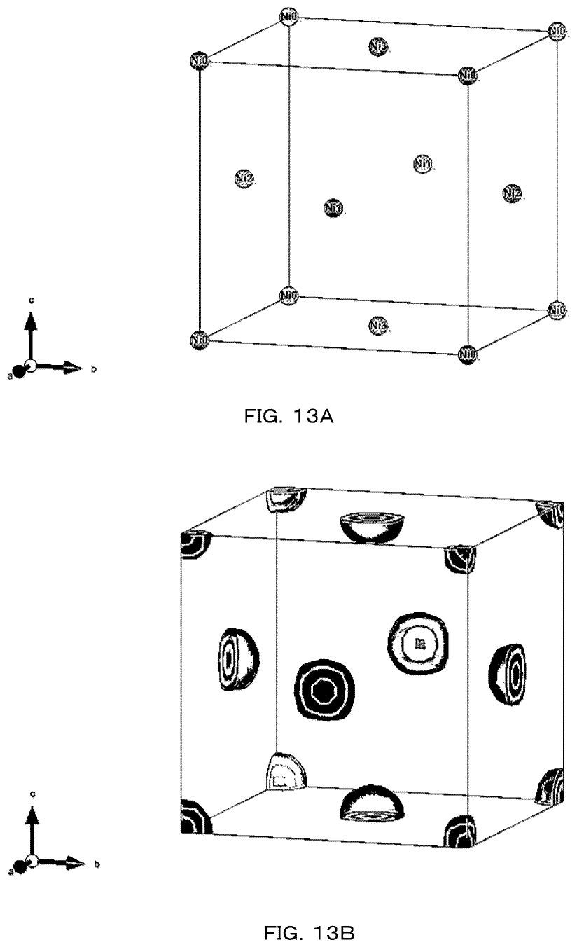

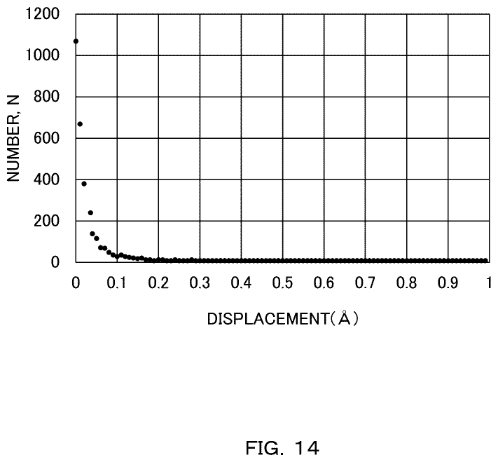

FIGS. 13A and 13B are diagrams showing statistics properties superimposed on the degenerate structural model of example 1, respectively. In FIG. 13A, spherical Ni atoms are arranged and displayed at the centroid positions. FIG. 13B shows the Volume data of the density distributions from the centroid positions in shading. FIG. 14 is a histogram showing the distribution of the average displacements from the centroid positions of all atoms in example 1. The average displacement from the centroid positions of all 2916 atoms in the post-calculation structural model was 0.022 Å, and the standard deviation of the average displacements was 0.035 Å. The method of the present disclosure can easily enable calculating such analytical values.

### Example 2

Total scattering data of x-quartz was measured in the same manner as in example 1. In addition, as in the first embodiment, the structural model was repeatedly generated by RMC so that the degree of deviation of S(Q) became smaller, and the structural model when the degree of deviation satisfied a predetermined condition was used as the post-calculation structural model. Then, the centroid position of the atoms of respective sites, Volume data of the density-distribution of the atoms of respective sites, the average displacement from the centroid positions of the atoms at respective sites, and the standard deviation of the average displacement were calculated.

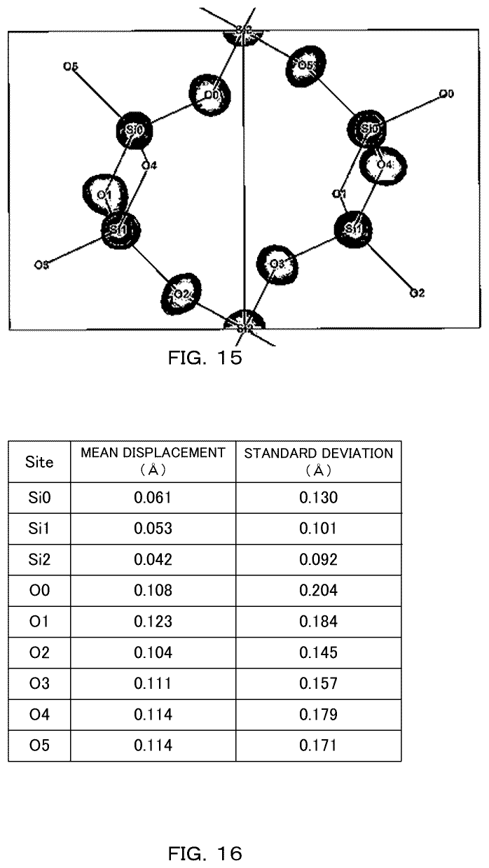

FIG. 15 is a diagram showing statistics properties superimposed on the degenerate structural model of example 2. FIG. 15 shows the centroid positions and the Volume data of the density-distribution at the respective atoms superimposed on the degenerate structural model. FIG. 16 is a table showing the average displacement and the standard deviation of the average displacements from the centroid positions of the atoms at the respective sites of example 2. According to FIG. 15 and FIG. 16, it can be seen that the average displacement from the centroid position is larger in O than in Si. Further, it can be seen that there is a difference in the average displacement from the centroid position for each site even in the same atom. The method of the present disclosure can also make such site-by-site features be confirmed numerically.

### Example 3

In the same manner as in example 1, the total scattering data of LCO (Lithium Cobalt Oxide) was measured. In addition, as in example 1, the structural model was repeatedly generated by RMC so that the degree of deviation of S(Q) became smaller, and the structural model when the degree of deviation satisfied a predetermined condition was used as the post-calculation structural model. Then, the centroid positions of the atoms of respective sites, Volume data of the density-distribution of the atoms of respective sites, the average displacement from the centroid positions of the atoms of respective sites, and the standard deviation of the average displacement were calculated.

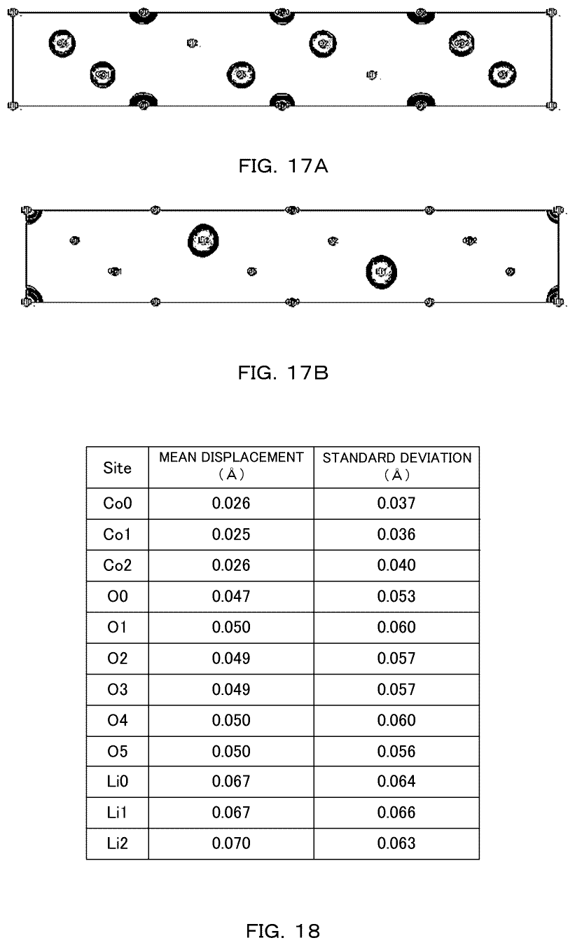

FIGS. 17A and 17B are diagrams showing statistics properties superimposed on the degenerate structural model of example 3. FIG. 17A shows the Volume data of the density distribution of Co and O and the centroid positions of respective atoms on the degenerate structural model. FIG. 17B shows the Volume data of the density distribution of Li and the centroid positions of respective atoms on the degenerate structural model. FIG. 18 is a table showing the average displacement from the centroid positions of atoms at respective sites and the standard deviation of the average displacement.

In the cathode material of Li ion battery and the all solid-state battery, both the framework structure and the distribution of Li ions are important. The method of the present disclosure is not limited to calculating and displaying the statistics properties of all elements, but also enables calculating and displaying the element information desired to be extracted.

According to the above-described results, the calculation apparatus, method, and program of the present disclosure can easily calculate the statistics properties such as the centroid position of the atoms or the sites of the structural model. Further, statistics properties such as the centroid position can be superimposed and displayed on the degenerate structural model.

Needless to say, the present disclosure is not limited to the above-described embodiments. The scope of the present disclosure covers various modifications and equivalents included in the technical idea of the present disclosure. In addition, the names, structures, shapes, numbers, positions, sizes, and the like of the constituent elements shown in the drawings are for convenience of explanation and may be changed as appropriate.

### DESCRIPTION OF SYMBOLS

10 calculation system
100 calculation apparatus
110 pre-calculation structural model acquiring section
120 pre-calculation information acquiring section
130 post-calculation structural model acquiring section
140 post-calculation information acquiring section
150 statistics property calculating section
160 simulation executing section
165 simulation condition setting section
170 degenerate structural model displaying section
175 statistics property displaying section
200 X-ray diffraction apparatus
210 X-ray generating section
220 incident side optical unit
230 goniometer
240 sample stage
250 emitting side optical unit
260 detector
300 control apparatus
310 control section
320 apparatus information storing section
330 measurement data storing section
340 display section
510 input device
520 display device

What is claimed is:

1. A calculation apparatus for calculating a statistics property of a structural model, comprising:
processing circuitry configured to
acquire a pre-calculation structural model in which atoms are periodically arranged,
acquire pre-calculation information that specifies atoms periodically arranged in the pre-calculation structural model,
acquire a post-calculation structural model calculated by using the pre-calculation structural model as source data,
acquire cluster information obtained by clustering atoms in the post-calculation structural model based on the pre-calculation information, and atom position information that is position information of atoms in the post-calculation structural model, and
calculate centroid positions of atoms having the same cluster information based on the cluster information and the atom position information.

2. The calculation apparatus according to claim 1, wherein the processing circuitry is further configured to
calculate the post-calculation structural model using the pre-calculation structural model as source data, and
acquire the calculated post-calculation structural model.

3. The calculation apparatus according to claim 2, wherein the processing circuitry is further configured to
calculate the post-calculation structural model by using Reverse Monte Carlo (RMC) or Molecular Dynamics (MD).

4. The calculation apparatus according to claim 3, wherein the processing circuitry is further configured to
set a size of a calculation cell in RMC or MD.

5. The calculation apparatus according to claim 1, wherein
the pre-calculation information is information of atomic coordinates in the pre-calculation structural model.

6. The calculation apparatus according to claim 1, wherein the processing circuitry is further configured to
calculate, in addition to the centroid position, displacement information regarding displacements of atoms having the same cluster information from the centroid positions based on the cluster information and the atom position information.

7. The calculation apparatus according to claim 1, wherein the processing circuitry is further configured to
display a degenerate structural model in which a unit structural model calculated by dividing the post-calculation structural model based on periodic information of the pre-calculation structural model is superimposed, and
display a statistics property on the degenerate structural model, wherein
the statistics property displayed on the degenerate structural model is the centroid position.

8. The calculation apparatus according to claim 6, wherein the processing circuitry is further configured to
display a degenerate structural model in which a unit structural model calculated by dividing the post-calculation structural model based on periodic information of the pre-calculation structural model is superimposed, and
display a statistics property on the degenerate structural model, wherein
the statistics property displayed on the degenerate structural model is the displacement information.

9. The calculation apparatus according to claim 7, wherein the processing circuitry is further configured to
display a plurality of the degenerate structural models in comparison, and
display the statistics properties corresponding to the respective degenerate structural models on them.

10. The calculation apparatus according to claim 8, wherein the processing circuitry is further configured to
display a plurality of the degenerate structural models in comparison,
display the statistics properties corresponding to the respective degenerate structural models, and
the statistics properties displayed on the respective degenerate structural models is the centroid position or the displacement information.

11. A method for calculating a statistics property of a structural model, comprising:
acquiring a pre-calculation structural model in which atoms are periodically arranged,
acquiring pre-calculation information that specifies atoms periodically arranged in the pre-calculation structural model,
acquiring a post-calculation structural model calculated by using the pre-calculation structural model as source data,
acquiring cluster information obtained by clustering atoms in the post-calculation structural model based on the pre-calculation information, and atom position information that is position information of atoms in the post-calculation structural model, and
calculating centroid positions of atoms having the same cluster information based on the cluster information and the atom position information.

12. A non-transitory computer-readable storage medium storing computer-readable instructions thereon which, when executed by a computer, cause the computer to perform a method, the method comprising:
acquiring a pre-calculation structural model in which atoms are periodically arranged,
acquiring pre-calculation information that specifies atoms periodically arranged in the pre-calculation structural model,
acquiring a post-calculation structural model calculated by using the pre-calculation structural model as source data,
acquiring cluster information obtained by clustering atoms in the post-calculation structural model based on the pre-calculation information, and atom position information that is position information of atoms in the post-calculation structural model, and
calculating centroid positions of atoms having the same cluster information based on the cluster information and the atom position information.
